(19) United States
(12) Patent Application Publication
(10) Pub. No.: US 2020/0315818 A1
(43) Pub. Date: Oct. 8, 2020

(54) Synergetic prosthesis

(71) Applicant: Ashvin Irrinki, Cupertino, CA (US)

(72) Inventor: Ashvin Irrinki, Cupertino, CA (US)

(21) Appl. No.: 16/773,757

(22) Filed: Jan. 27, 2020

Related U.S. Application Data
(63) Continuation-in-part of application No. 15/976,871, filed on May 11, 2018, now Pat. No. 10,688,702.

(51) Int. Cl. A61F 2/72; A61F 2/64; A61F 2/66; A61F 2/70

(52) U.S. Cl. CPC A61F 2/72; A61F 2/64; A61F 2002/5001; A61F 2/70; A61F 2002/705; A61F 2/6607

(57) ABSTRACT

The synergetic prosthetic system comprises of a knee and an ankle sleeve on the functional leg and a prosthesis on the amputated leg. Each sleeve comprises of a power supply, an accelerometer, a wireless transmitter and a wire connecting the accelerometer to the wireless transmitter. The prosthesis comprises of a power supply, a microcontroller, servo motors, a knee joint assembly, and an ankle joint assembly. A steel rod covered with a casing connects the knee and ankle joints. The accelerometer measures and transmits the angle of rotation of the functional knee and ankle joint to a microcontroller. The microcontroller pre-programmed with normal gait data pairs the data from the healthy leg and controls the servo motors. The servo motors, move the prosthetic joints to mimic a normal human gait.

### CROSS REFERENCES TO RELATED APPLICATIONS

Not Applicable

### FEDERALLY SPONSORED RESEARCH OR DEVELOPMENT

Not Applicable

### BACKGROUND

### Field of the Invention

The present invention relates generally to a prosthetic device for single-leg above the knee amputees. More particularly, the synergetic prosthesis system utilizes signals from the position sensors on the healthy leg and preprogrammed gait data stored in a microcontroller to move the prosthesis in synergy to simulate near normal human gait.

### Description of Related Art

Over the years, humans with an inability to walk due to amputations relied on prosthetics to serve their most basic needs. Vital limbs, such as legs, now have prosthetics that can efficiently compensate for the lost limb. The first prosthetics were simply wooden pegs relying on limbs above the amputated region to move. Prosthetics have managed to escalate in design and functionality over time. Modern-day prosthetics advanced from simple wooden mechanism to intricate and elaborate designs that actually aid in the normal human gait. However, these prosthetics are solely responsible to provide comfort to the amputees, despite having to mimic something as intricate as the normal human gait. Current prosthetics rely on repetitive and simple mechanisms such as the use of momentum or hydraulics in order to propel the limb forward that try their best to replicate the normal human gait, but fail to do so. Others use motors, but receive information from the brain waves. Current prosthetics lack control from the user and prevent the amputees from normal functional gait pattern. In the prior art, the prosthetic industry is continuously making strides to mimic normal human functional gait pattern.

Based on recent studies, prosthetics that fail to comply with the intricacies of the normal human gait causes unwarranted problems, including back pain. Researchers have tackled this issue and were partially successful, as they created a prosthesis that harnesses brain waves from the patient in order to control the prosthetic. As promising as this prosthetic may be, the costs to obtain one is appalling. Although prosthetic design has advanced in recent years, the prior art still has limitations to meet the functional needs of the amputees. There exists an extensive opportunity for design advancements and innovation where the prior art fails or is deficient. It is therefore an object of the present invention to mitigate these hindrances.

### SUMMARY

In general, the present invention of a synergetic prosthesis for single-leg above the knee amputees provides normal human gait pattern where the prior art fails. The present invention generally will improve an amputees gait by receiving electronic signals from the healthy functional leg, compare the signals with pre-programmed gait analysis data and transmit signals to the prosthetic to allow the amputees gait to be reminiscent of a normal human gait. This prosthesis involves work in three major fields, the electrical, mechanical and the medical fields which contributes to its difficulty. The mechanical aspect involves the design and construction of a viable prosthetic. The electrical aspect involves the programming and the writing of the prosthetic. The medical aspect involves understanding the human gait study.

The human gait is the mapped motion of the precise way we walk. Dynamic gait evaluation provides intrinsic and extrinsic factors affecting an individual's ability to walk. The evaluation also provides range of motion or kinematics such as positions, angles, velocities, and accelerations of body segments and joints during motion. In addition, kinetics or forces that cause the body to move are evaluated using electromyogram (EMG) tracing of the muscle activity. This information is widely published and readily available for use in the design of the prosthetics. The present invention uses this data to move the prosthesis in synergy with the healthy leg to mimic the normal human gait.

Over the course of countless studies, scientists have narrowed an entire stride of human gait into two main phases, the Stance Phase and the Swing Phase. There are multiple sub phases in each main phase. The Stance Phase contributes to 60% of the stride which includes Initial Contract (heel strike), Loading Response (foot flat), Mid Stance, Terminal Stance (heel off), and Pre-Swing (toe-off). The Swing Phase contributes to 40% of the stride which includes Initial Swing (acceleration), Mid Swing, and Terminal Swing (deceleration). The prosthetics that do not follow the human gait have been proven to cause back and foot pain. In order to eliminate discomfort, the aim of this prosthetic is to model the human gait as precisely and accurately as possible. This prosthetic is strictly for patients who have a functional and healthy leg, but require a prosthesis for the other leg.

The key design of the synergetic prosthesis is to follow the human gait cycle by receiving and interpreting angle of rotation of the knee and ankle joints from the healthy leg and move the prosthetic to corresponding position to mimic the human gait. To achieve this, the use of accelerometers to properly measure the position is necessary.

An accelerometer is a sensor that measures the acceleration in three directions by measuring the change in capacitance between a microelectromechanical spring-mass system. The change in acceleration in the x, y, and z directions are measured by the sensor with this system. The accelerometers will be placed on the shin and the foot of the functional leg, to measure the knee and ankle joints, respectively. Two servo motors will be placed on the prosthetic knee and the ankle at the same position as the healthy leg to achieve the desired synergy. The accelerometers will measure the angle of rotation of the knee and ankle, and wirelessly send this data to the microcontroller. The microcontroller is pre-programmed with the normal gait cycle data to aid in the determination of the position of the prosthetic joints. For description purposes, in the normal human gait, the right knee angle is labelled ANGLE 1 and the left knee angle is labelled ANGLE 2. Similarly, the right ankle angle is labelled ANGLE 3, and the left ankle angle is labelled ANGLE 4. For instance, if the left leg is amputated, ANGLE 2 and 4 will be represented on the prosthetic leg. This means that the microcontroller will pair ANGLE 1 with ANGLE 2 and ANGLE 3 with ANGLE 4. In other words, if the accelerometer reads ANGLE 1 on the healthy knee, it will send the data to the micro-controller, which will pair it with ANGLE 2 on the prosthetic leg. Then, the microcontroller signals the servo motor to move to ANGLE 2. The servo motors will cause the prosthesis joints to move accordingly, synchronous to the healthy leg.

The results conclude that it is possible to use the joint angles of the healthy leg to move the prosthetic knee and ankle joints. Though not as appealing, the design and construction of this prosthetic allows the amputees to have proper mobility and comfort, as it follows human gait.

This prosthetic can help thousands of people with lower limb amputations. By successfully improving the design and materials, this prosthetic will be very useful and aid many people.

The disclosed embodiments are illustrative, not restrictive. While specific configurations of the prosthetic have been described, it is understood that the present invention can be applied to a wide variety of prosthesis. There are many alternative ways of implementing the invention.

These and other features, aspects, and advantages of the present invention will become better understood with reference to the following description and claims.

### BRIEF DESCRIPTION OF THE DRAWINGS

Note: For simplicity of illustration, bearings, nuts, bolts, washers, and minutiae of common prosthesis industry hardware are not depicted as they are known to those with skill in the art. When they are shown, it is purely for illustrative purposes and not intended to capture all embodiments of the invention disclosed.

FIG. 1 is a perspective assembled view of the knee and ankle sleeves.

FIG. 2 is an exploded view of the knee sleeve shown in FIG. 1.

FIG. 3 is an exploded view of the ankle sleeve shown in FIG. 1.

FIG. 4 is a perspective assembled view of the synergetic prosthesis with the knee and the ankle joint

FIG. 5 is an exploded view of the prosthetic knee and the ankle joint shown in FIG. 4.

FIG. 6 is a block diagram showing an example of a control system for the synergetic prosthetic system as shown in FIG. 1 and FIG. 4.

FIG. 7 is a diagram of a prosthesis system in accordance with certain embodiments.

FIG. 8 is a diagram illustrating tilt sensing using a single axis accelerometer.

FIG. 9 is a graph showing output acceleration versus angle of inclination for single axis inclination sensing.

FIG. 10 is a graph showing incremental inclination sensitivity for one-degree steps.

FIG. 11 is a graph showing incremental inclination sensitivity for quarter-degree steps.

FIG. 12 is a graph showing a comparison of inverse sine function and linear approximation for inclination angle calculation.

FIG. 13 is a graph showing calculated angle error for different scaling factors.

FIG. 14 is a diagram illustrating tilt sensing using a two axis accelerometer.

FIG. 15 is a graph showing output acceleration versus angle of inclination for dual-axis inclination sensing.

FIG. 16 is a graph showing minimum accelerometer resolution for a desired angle of inclination resolution.

FIG. 17 is a diagram illustrating angle of inclination and sign of acceleration for quadrant detection.

FIG. 18A is a diagram illustrating angle of spherical coordinate system.

FIG. 18B is a diagram illustrating angle of spherical coordinate system.

FIG. 18C is a diagram illustrating angle of spherical coordinate system.

FIG. 18D is a diagram illustrating angle of spherical coordinate system.

FIG. 19A is a diagram illustrating angle for independent inclination sensing.

FIG. 19B is a diagram illustrating angle for independent inclination sensing.

FIG. 19C is a diagram illustrating angle for independent inclination sensing.

FIG. 19D is a diagram illustrating angle for independent inclination sensing.

FIG. 20 is a graph showing calculated angle error due to accelerometer offset.

FIG. 21 is a graph showing calculated angle error due to accelerometer sensitivity mismatch.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

a. Accelerometer—a sensor that reads the angle of rotation while measuring the acceleration forces
b. Microcontroller—a control board that allows various types of input/output devices to interface with the board
c. Servo motor—is a rotary actuator or linear actuator that allows for precise control of angular or linear positions, velocity and acceleration
d. Synergy—“the interaction or cooperation of two or more organizations, substances, or other agents to produce a combined effect greater than the sum of their separate effects.”
e. Human Gait Cycle—the precise, mapped cycle of the way humans walk “Human gait refers to locomotion achieved through the movement of human limbs”

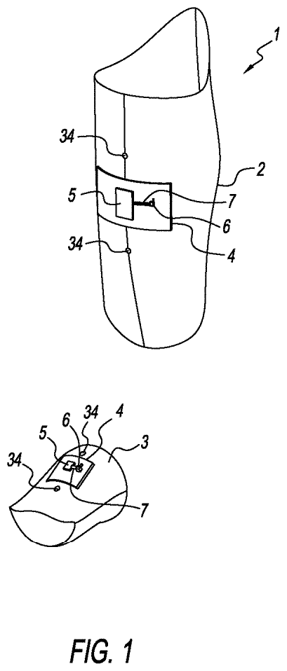

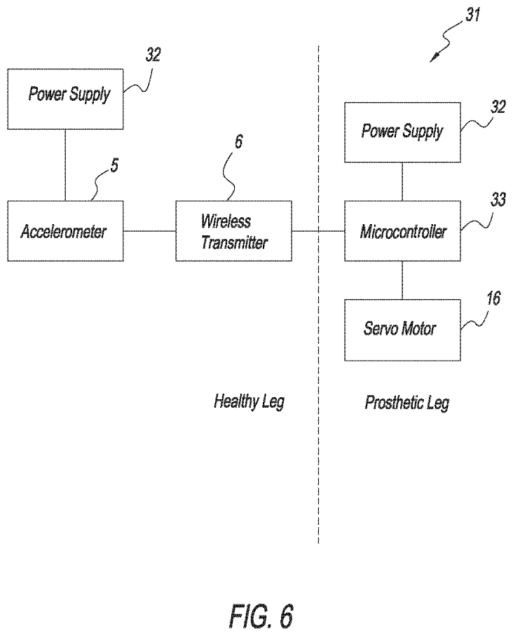

In FIG. 1, a perspective view of the knee and ankle sleeves (1) of the healthy leg are shown. A flexible, polyester knee sleeve (2) will slide over the patient's shin. An aluminum plate (4) is placed on the sleeve (2) to hold an accelerometer (5), and a wireless transmitter (6). A power supply (32), as shown in FIG. 6, supplies power to the accelerometer (5) and the wireless transmitter (6). The accelerometer (5) reads the angle of rotation when the knee is moving. To ensure the accelerometer (5) measure the same angles and readings for each stride the amputee takes, two positional markers (34) on the knee sleeve (2) are used to properly match with the position markers on the shin. A connecting wire (7) transfers the readings from the accelerometer to the wireless transmitter (6). The wireless transmitter (6) transmits the readings to the microcontroller (33), shown in FIG. 6. Another flexible, polyester foot sleeve (3) will slide over the patient's foot and has the same components and purpose as the knee sleeve (2). An aluminum plate (4) is placed on the foot sleeve (3) to hold an accelerometer (5), and a wireless transmitter (6). A power supply (32), as shown in FIG. 6, supplies power to the accelerometer (5) and the wireless transmitter (6). The accelerometer (5) reads the angle of rotation when the ankle is moving. To ensure the accelerometer (5) measures the same angles and readings for each stride the amputee takes, two positional markers (34) on the foot sleeve (2) are used to properly match with the position markers on the foot. The connecting wire (7) transfers the readings from the accelerometer to the wireless transmitter (6). The wireless transmitter (6) transmits the readings to the microcontroller (33), shown in FIG. 6.

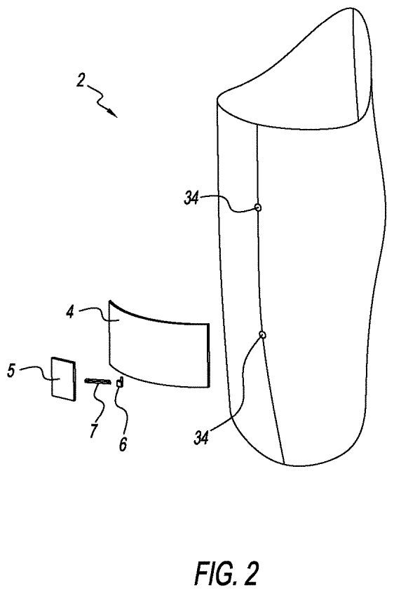

In FIG. 2, an exploded view of the knee sleeve (2) is shown. The accelerometer (5), the connecting wire (7), the wireless transmitter (6), the position markers (34), and the aluminum plate (4) can be seen in detail.

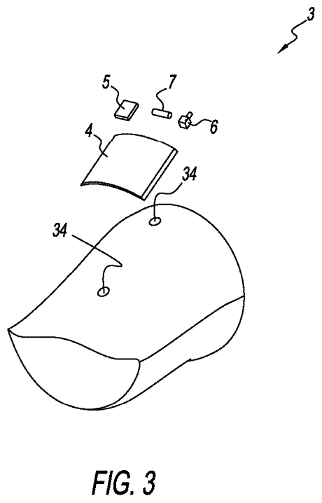

In FIG. 3, an exploded view of the ankle sleeve (3) is shown. The accelerometer (5), the connecting wire (7), the wireless transmitter (6), the position markers (34), and the aluminum plate (4) can be seen in detail.

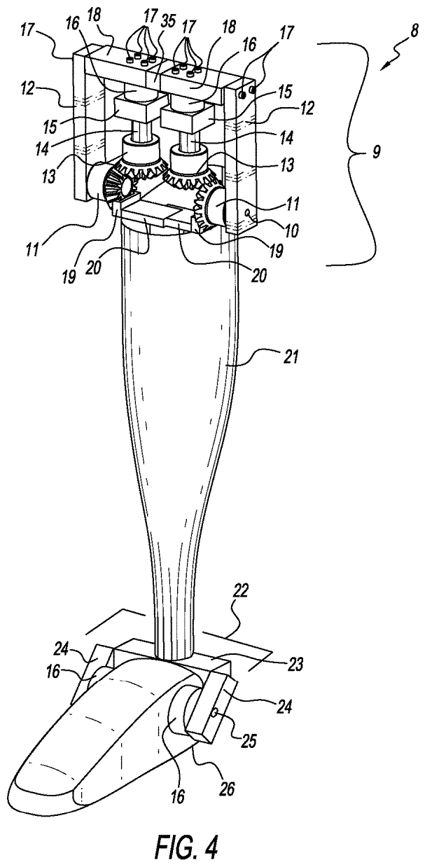

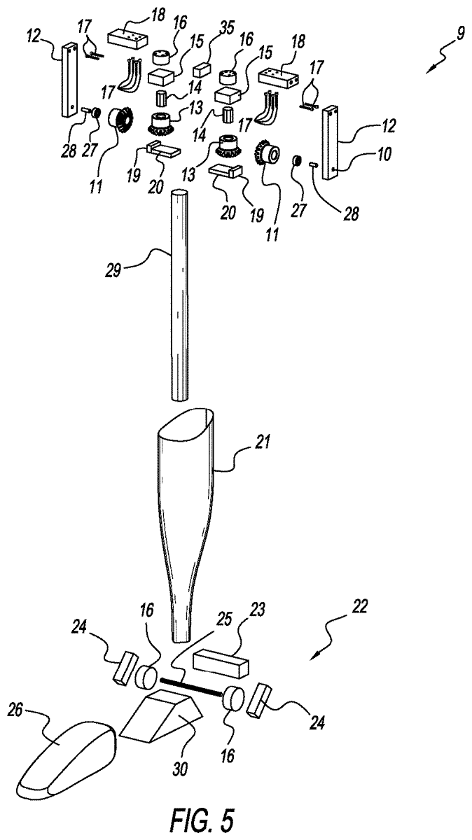

In FIG. 4, a perspective view of the synergetic prosthesis (8) is shown. The prosthetic knee joint (9) is responsible for moving the leg forwards and backwards. In order to do this, two servo motors (16) are connected. They are mounted by 4 screws (17) to a horizontal support member (18). The two servo motors (16) are placed vertically to conserve space in the vertical direction. As the two servo motors (16) need to move the entire leg, a gearbox (15) is needed to increase the torque the servo motor (16) produce. A metal shaft (14) is used to transfer the motion to an inverted gear (13). As the servo motors (16) is produce motion in the horizontal direction, a second gear (11) is needed to convert the horizontal motion to vertical motion. This gear (11) contains a steel ball bearing (27) for a smoother motion. A small, steel shaft (28), as shown in FIG. 5, is used to connect this gear (11) and the bearing (27) to a vertical support member (12). To secure this shaft (28), a sleeve is inserted into the vertical member (12). This vertical support member (12) is connected to the horizontal support member (18) by 2 identical screws (17). In order to move the leg, the bottom of the gear (11) is connected to a small, vertical member (19) which is connected to a small, horizontal member (20). This member (20) is attached to a steel rod (29), as shown in FIG. 5. A strong, durable, metal casing (21) covers this steel rod. The base of the steel rod (29) is connected to another horizontal support member (23) which is the beginning of the prosthetic foot. The prosthetic ankle joint (22) contains the support member (23), which is connected to the steel rod (29). The ankle joint (22) moves independently of the knee joint (9). The support member (23) is connected to two other diagonal support members (24). These members (24) hold two servo motors (16) that move the foot. A metal studding (25), as shown in FIG. 4, connects the two motors to a copper foot (30), as shown in FIG. 6. This copper foot is covered by a metal casing (26).

FIG. 5 shows an exploded view of the synergetic prosthesis (8) mentioned in FIG. 4. The exploded view of the knee joint (9) and the exploded view of the ankle joint (22) is shown. The steel shaft (28), the ball bearing (27), the screws (17), the vertical steel rod (29), the foot horizontal support member (23), the diagonal support members (24) that hold the motors (16), the studding (25), the copper foot (30), and the metal casing (26) can be clearly seen. In addition, (35) is a spacer used to connect the two horizontal support members (18) to prevent the gears from interlocking.

FIG. 6 depicts a block diagram of the control system (31). The power supply (32) provides power for the accelerometers (5) and the wireless transmitters (6). The two accelerometers (5) reads the angle of rotation for both the knee and the ankle and the wireless transmitters (6) sends the signals to the microcontroller (33). The microcontroller controls the two servo motors (16) based on the angles the accelerometer reads. Another power supply (32) provides power to the microcontroller (33) and the servo motors (16).

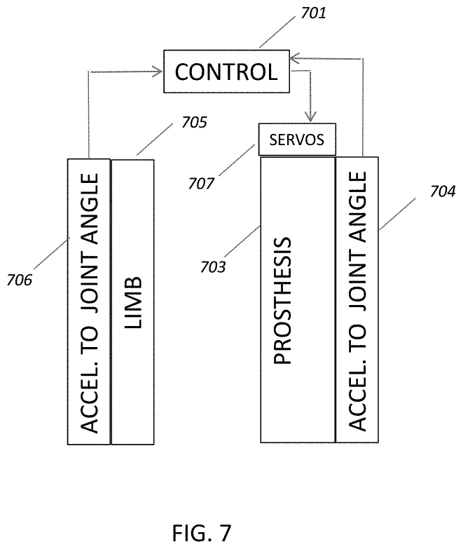

Referring to FIG. 7, a diagram is shown of a prosthesis system in accordance with certain embodiments. In these embodiments, a controller 701 is used to drive a prosthesis 703 in accordance with movement of a healthy limb 705. Sensors (in this case, accelerometers) are fitted to the prosthesis 703 and the health limb 705. Accelerometer data may be converted to joint angle data in blocks 704 and 706 a manner described hereafter. The controller 701 controls servos 707 to drive the prosthesis 703 in accordance with movement of the healthy limb 705 such that the movement of the prosthesis mimics that of the healthy limb.

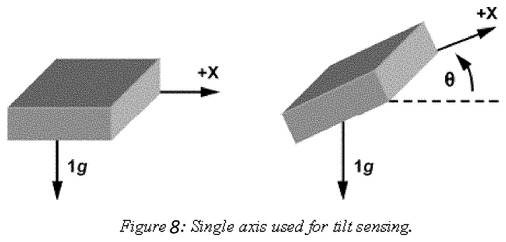

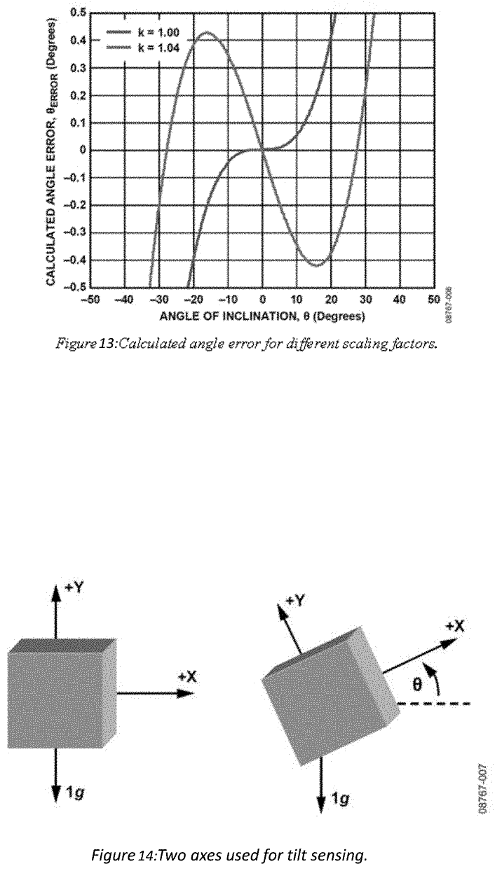

Accelerometer data may be converted to joint angle data, for example, in a manner described in Using An Accelerometer far Inclination Sensing by Christopher J. Fisher, Convergence Promotions LLC, 2011-05-06 (referred to hereafter as “Fisher”), incorporated hereby by reference. The following description, which makes reference to FIGS. 8-21, is taken from Fisher.

In suitable applications, where the net acceleration or force on a system over time is gravity, an accelerometer can be used to measure the static angle of tilt or inclination. Such applications include gaming, horizon detection in digital cameras, and detecting the heading of a device in industrial and medical applications.

The underlying assumption in inclination sensing with an accelerometer is that the only acceleration stimulus is that associated with gravity. In practice, signal processing can be performed on the signal output to remove high frequency content from the output signal, so some AC acceleration can be tolerated.

Inclination sensing uses the gravity vector, and its projection on the axes of the accelerometer, to determine the tilt angle. Because gravity is a DC acceleration, any forces that result in an additional DC acceleration corrupt the output signal and result in an incorrect calculation. Sources of DC acceleration include the period of time when a vehicle is accelerating at a constant rate and rotating devices that induce a centripetal acceleration on the accelerometer. In addition, rotating an accelerometer through gravity causes an apparent AC acceleration as the projection of gravity on the axes of interest changes. Any filtering of the acceleration signal before calculating the inclination affects how quickly the output settles to the new static value.

In applications where inclination sensing is needed only over a limited angle, and with a somewhat coarse resolution, a single-axis device (or a single axis of a multiple-axis device) can be used.

For example, in FIG. 8 a single axis (the x-axis in this example) is rotated through gravity. Because this approach uses only a single axis and requires the gravity vector, the calculated angle of inclination is accurate only when the device is oriented so that the x-axis is always in the plane of gravity. Any rotation about the other axes reduces the magnitude of the acceleration on the x-axis and results in error in the calculated angle of inclination.

Referring to basic trigonometry, the projection of the gravity vector on the x-axis produces an output acceleration equal to the sine of the angle between the accelerometer x-axis and the horizon. The horizon is typically taken to be the plane orthogonal to the gravity vector. For an ideal value of 1 g for gravity, the output acceleration is:

A_{X,OUT}[g]=1g×sin(θ) (1)

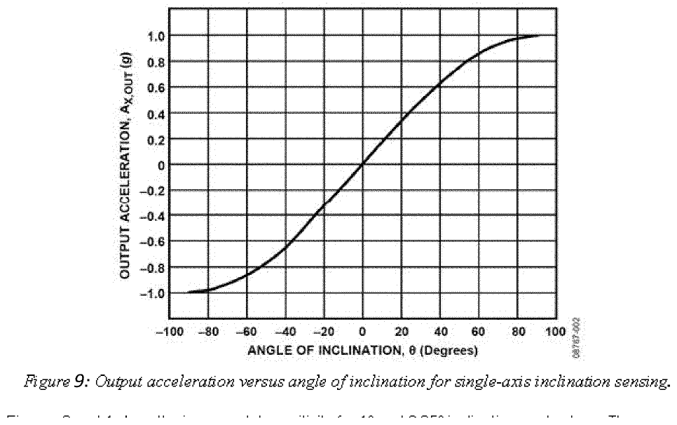

When using a single-axis solution, note that the sensitivity—that is, the change in output for some change in input—of the inclination calculation decreases as the angle between the horizon and the x-axis increases, approaching zero as the angle approaches ±90°. This can be seen in FIG. 9, where the output acceleration, in g, is plotted against the angle of inclination. Near ±90°, a large change in inclination angle results in a small change in output acceleration.

Because the inclination calculation is done digitally, the output acceleration is presented as a constant acceleration per least significant bit (LSB) or code, obtained either from an analog-to-digital converter (ADC) or directly from a digital output part. Because the output resolution is a constant acceleration, the resolution in degrees of inclination is variable, with the best resolution close to 0° and the worst resolution at ±90°.

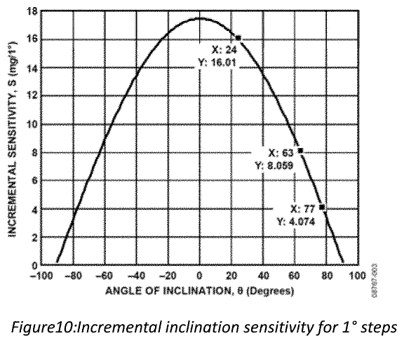

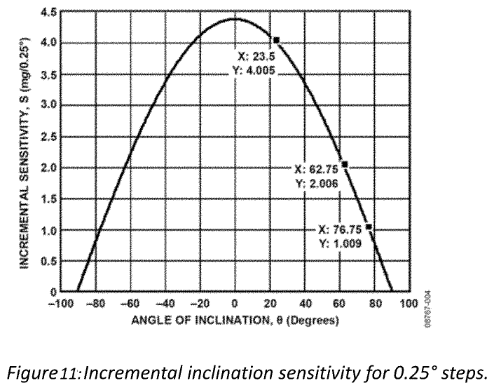

FIGS. 10 and 11 show the incremental sensitivity for 1° and 0.25° inclination angle steps. The incremental sensitivity is the output change, shown in mg, per inclination angle step, or

S[g]=1g×(sin(N+P)−sin(N)) (2)

where:

N is the current angle.

P is the step size.

These curves can be used to determine the minimum necessary resolution when measuring the output acceleration in order to meet the desired inclination resolution for the entire range of an application. For example, designing for a maximum step size of 1°, a resolution of at least 8 mg/LSB is necessary for a range of ±63°. Similarly, to achieve a maximum step size of 0.25° for a range of ±63° requires a resolution of at least 2 mg/LSB. Note that, if enough either is present, oversampling can be used to achieve better resolution.

Because the output of the accelerometer obeys a sinusoidal relationship as it is rotated through gravity, conversion from acceleration to angle is done using the inverse sine function,

$$θ = {sin}^{- 1} ( \frac{{A}_{X , OUT} [ g ]}{1 g} ) \qquad ( 3 )$$

where the inclination angle, θ, is in radians.

If a narrow range of inclination is required, a linear approximation can be used in place of the inverse sine function. The linear approximation relates to the approximation of sine for small angles,

sin(θ)≅θ,θ<<1 (4)

where the inclination angle, θ, is in radians.

$$θ \tilde{=} k \times ( \frac{{A}_{X , OUT} [ g ]}{1 g} ) \qquad ( 5 )$$

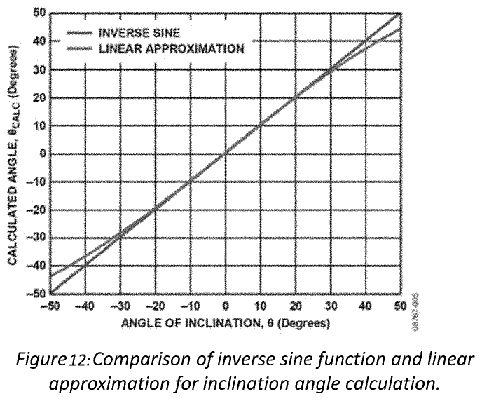

Conversion to degrees is done by multiplying the result of Equation 5 by (180/π) FIG. 12 shows a comparison between using the inverse sine function and the linear approximation with k equal to 1. As the magnitude of the inclination angle increases, the linear approximation begins to fail, and the calculated angle deviates from the actual angle.

Because the calculated angle is plotted against the actual angle of inclination, the linear approximation appears to bend near the ends. This is because the linear approximation is linear only when compared to the output acceleration and, as shown in FIG. 9, the output acceleration behaves similarly as the actual angle of inclination is increased. However, the inverse sine function should produce an output that is one-to-one with the actual angle of inclination, causing the calculated angle to be a straight line when plotted against the actual angle of inclination.

As an example, if the desired resolution of inclination sensing is 1°, an error of ±0.5° is acceptable because it is below the rounding error of the calculation. If the error between the actual angle of inclination and the calculated angle of inclination is plotted fork equal to 1, as shown in FIG. 6, the valid range for the linear approximation is only ±20°. If the scaling factor is adjusted such that the error is maximized, but kept within the calculation rounding limits, the valid range of the linear approximation increases to greater than ±30°.

### Dual-Axis Tilt Calculation

One limitation of single-axis inclination sensing is the need for a high-resolution ADC or digital output to achieve a large range of valid inclination angles, as shown in FIGS. 10 and 11. Another limitation is that a single-axis measurement cannot provide a 360° measurement, because the acceleration generated at an inclination of N° is the same as the acceleration generated at an inclination of 180°−N°.

For some applications this is acceptable, but for applications that require higher resolution, or the ability to distinguish angles of inclination in a complete 360° arc, a second axis, as shown in FIG. 14, or a second sensor is necessary. If a second sensor is used, it should be oriented such that the sensing axis of the first sensor.

There are three major benefits to including a second axis in determining the angle of inclination. These benefits are described in the following sections.

Constant Sensitivity

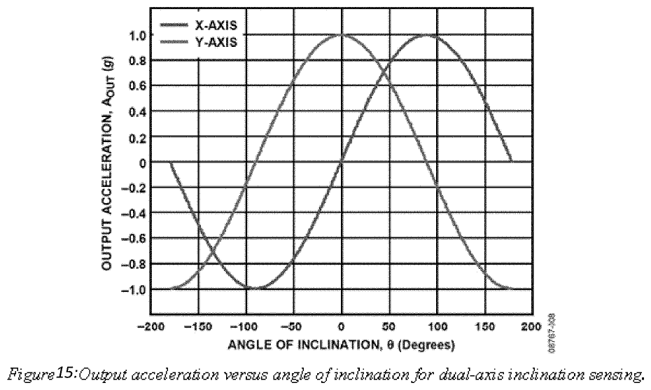

The first major benefit of using a second axis is due to the orthogonality of the axes. As in the single-axis solution, the acceleration detected by the x-axis is proportional to the sine of the angle of inclination. The y-axis acceleration, due to the orthogonality, is proportional to the cosine of the angle of inclination (see FIG. 15). As the incremental sensitivity of one axis is reduced, such as when the acceleration on that axis approaches +1 g or −1 g, the incremental sensitivity of the other axis increases.

One method to convert the measured acceleration to an inclination angle is to compute the inverse sine of the x-axis and the inverse cosine of the y-axis, similar to the single-axis solution. However, an easier and more efficient approach is to use the ratio of the two values, which results in the following:

$$\begin{matrix}\frac{{A}_{X , OUT}}{{A}_{Y , OUT}} = \frac{1 g \times sin ( θ )}{1 g \times cos ( θ )} = tan ( θ ) & ( 6 ) \\ θ = {tan}^{- 1} ( \frac{{A}_{X , OUT}}{{A}_{Y , OUT}} ) & ( 7 )\end{matrix}$$

where the inclination angle, θ, is in radians.

Unlike the single-axis example, using the ratio of the two axes to determine the angle of inclination makes determining an incremental sensitivity very difficult. Instead, it is more useful to determine the minimum necessary accelerometer resolution, given a desired inclination resolution. Given that the incremental sensitivity of one axis increases as the other decreases, the net result is an effective incremental sensitivity that is roughly constant. This means that the selection of an accelerometer with sufficient resolution to achieve the desired inclination step size at one angle is sufficient for all angles.

To determine the minimum necessary accelerometer resolution, Equation 6 is examined to determine where the resolution limitations are. Because the output of each axis relies on the sine or cosine of the angle of inclination, and the angle of inclination for each function is the same, the minimum resolvable angle corresponds to the minimum resolvable acceleration.

As shown in FIGS. 10 and 11, the sine function has the greatest rate of change near 0°, and the cosine function has the least rate of change at this point. For this reason, the change in acceleration on the x-axis due to a change in inclination is recognized before a change in acceleration on the y-axis. Therefore, the resolution of the system near 0° depends primarily on the resolution of the x-axis. To detect an inclination change of P°, the accelerometer must be able to detect a change of approximately:

ΔA_{OUT}[g]≅1g×sin(P) (8)

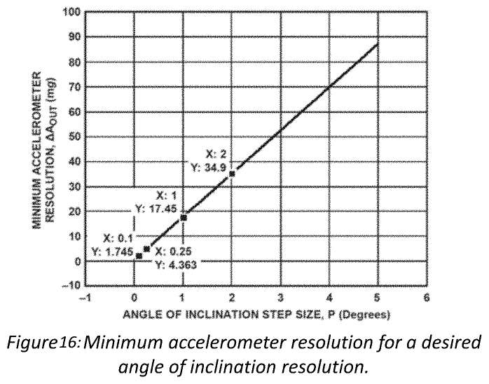

FIG. 16 can be used to determine the minimum necessary accelerometer resolution—or maximum accelerometer scale factor—for a desired inclination step size. Note that increased accelerometer resolution corresponds with a reduction in accelerometer scale factor and with the ability to detect a smaller change in output acceleration. Therefore, when selecting an accelerometer with the appropriate resolution, the scale factor should be less than the limit shown in FIG. 16 for the intended inclination step size.

Reduced Dependence on Alignment with Plane of Gravity

The second major benefit of using at least two axes is that unlike the single-axis solution, where tilt in any axis other than the x-axis can cause significant error, the use of a second axis allows for an accurate value to be measured even when inclination in the third axis is present. This is because the effective incremental sensitivity is proportional to the root-sum-square (RSS) value of gravity on the axes of interest.

When gravity is completely contained in the xy-plane, the RSS value of acceleration detected on those axes is ideally equal to 1 g. If tilt is present in the xz- or yz-plane, the total acceleration due to gravity is reduced, which also reduces the effective incremental sensitivity. This, in turn, increases the inclination step size for a given accelerometer resolution, but still provides an accurate measurement. The resulting angle from the inclination calculation corresponds to the rotation in the xy-plane.

If the system is tilted enough, such that very little acceleration due to gravity is present in the xy-plane, the inclination angle step size will be too coarse to be useful. Therefore, it is recommended that tilt in the xz- or yz-plane be limited.

Complete 360° Tilt Sensing

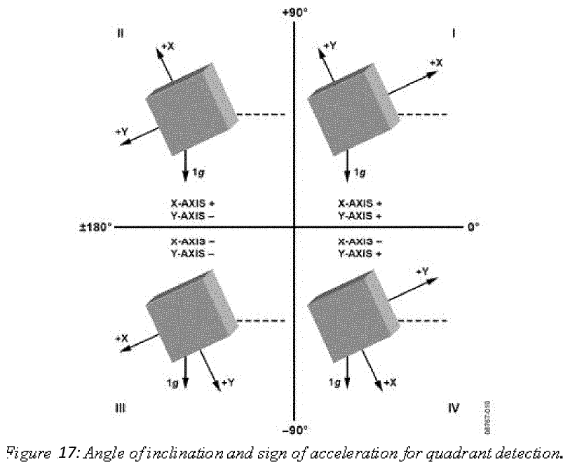

The third major benefit of using a second axis is the ability to distinguish between each quadrant and to measure angles throughout the entire 360° arc. As shown in FIG. 17, each quadrant has a different combination of signs associated with the x- and y-axis acceleration.

The inverse tangent function returns a value in Quadrant I if the operand, AX,OUT/AY,OUT, is positive; if the operand is negative, the inverse tangent function returns a value in Quadrant IV. Because the operand in Quadrant II is negative, a value of 180° should be added to the result of the calculation when the angle is in that quadrant.

Because the operand in Quadrant III is positive, a value of 180° should be subtracted from the result of the calculation when the angle is in that quadrant. The correct quadrant of the calculated angle can be determined by examining the sign of the measured acceleration on each axis.

Triple-Axis Tilt Calculation

When a third axis is introduced, the orientation of the sensor can be determined in a complete sphere. The classical method of rectangular (x, y, z) to spherical (ρ, θ, φ) conversion can be used to relate the angle of tilt in the xy-plane, θ, and the angle of inclination from the gravity vector, φ, to the measured acceleration in each axis, as follows:

$$\begin{matrix}θ = {tan}^{- 1} ( \frac{{A}_{X , OUT}}{{A}_{Y , OUT}} ) & ( 9 ) \\ φ = {cos}^{- 1} ( \frac{{A}_{Z , OUT}}{\sqrt{{A}_{X , OUT}^{2}} + {A}_{Y , OUT}^{2} + {A}_{Z , OUT}^{2}} ) & ( 10 )\end{matrix}$$

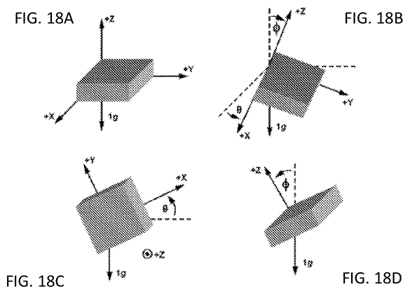

Given the assumption that the only measured acceleration is due to gravity, the denominator of the operand in Equation 10 can be replaced with a constant, ideally 1, because the RSS value of all the axes is constant when the only acceleration is gravity. The angles are shown in FIG. 18, where FIG. 18c shows θ only in the xy-plane, and FIG. 18d shows φ as the angle between the z-axis and the gravity vector.

Due to the similarities between the equations for the triple-axis method and the equations for the single- and dual-axis methods, the analysis of the triple-axis solution is the same as for the single- and dual-axis methods combined. The measurement of θ benefits from the ratio of two orthogonal axes, and a desired inclination resolution requires a minimum accelerometer resolution as described by Equation 8.

The measurement of φ corresponds to the measurement of the inclination angle for the single-axis solution, along with the method for determining the minimum accelerometer resolution needed for a specific inclination angle resolution over a desired range. The difference is the use of the inverse cosine function to determine φ results in a maximum incremental sensitivity, when φ is 90° and a minimum incremental sensitivity at 0° and 180°.

A plot similar to FIGS. 10 and 11 can be generated by substituting cosine for sine in Equation 2. It is important to note that although θ ranges from −180° to +180°, φ ranges only from 0° to 180°. A negative angle for φ causes the angle of θ to become negative.

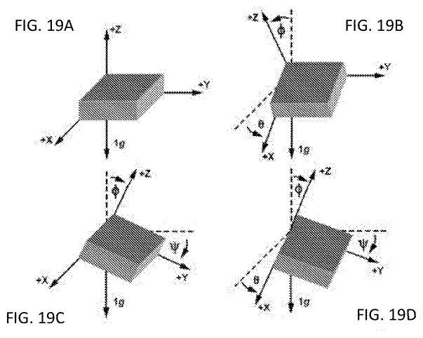

An alternative method for inclination sensing with three axes is to determine the angle individually for each axis of the accelerometer from a reference position. The reference position is taken as the typical orientation of a device, with the x- and y-axes in the plane of the horizon (0 g field) and the z-axis orthogonal to the horizon (1 g field). This is shown in FIG. 19, with θ as the angle between the horizon and the x-axis of the accelerometer, ψ as the angle between the horizon and the y-axis of the accelerometer, and φ as the angle between the gravity vector and the z-axis. When in the initial position of 0 g on the x- and y-axes and 1 g on the z-axis, all calculated angles would be 0°.

Basic trigonometry can be used to show that the angles of inclination can be calculated using Equation 11, Equation 12, and Equation 13.

$$\begin{matrix}θ = {tan}^{- 1} ( \frac{{A}_{X , OUT}}{\sqrt{{A}_{X , OUT}^{2} + {A}_{Z , OUT}^{2}}} ) \\ & ( 11 ) \\ ψ = {tan}^{- 1} ( \frac{{A}_{Y , OUT}}{\sqrt{{A}_{X , OUT}^{2} + {A}_{Z , OUT}^{2}}} ) & ( 12 ) \\ φ = {tan}^{- 1} ( \frac{\sqrt{{A}_{X , OUT}^{2} + {A}_{Y , OUT}^{2}}}{{A}_{Z , OUT}} ) & ( 13 )\end{matrix}$$

The apparent inversion of the operand in Equation 13 is due to the initial position being a 1 g field. If the horizon is desired as the reference for the z-axis, the operand can be inverted. A positive angle means that the corresponding positive axis of the accelerometer is pointed above the horizon, whereas a negative angle means that the axis is pointed below the horizon.

Because the inverse tangent function and a ratio of accelerations are used, the benefits mentioned in the dual-axis example apply, namely that the effective incremental sensitivity is constant, and that the angles can be accurately measured for all points around the unit sphere.

Calibration for Offset and Sensitivity Mismatch Error

The foregoing analysis was done under the assumption that an ideal accelerometer was used. This corresponds to a device with no 0 g offset and with perfect sensitivity (expressed as mV/g for an analog sensor or LSB/g for a digital sensor). Although sensors come trimmed, the devices are mechanical in nature, which means that any static stress on the part after assembly of the system may affect the offset and sensitivity. This, combined with the limits of factory calibration, can result in error beyond the allowable limits for the application.

Effects of Offset Error

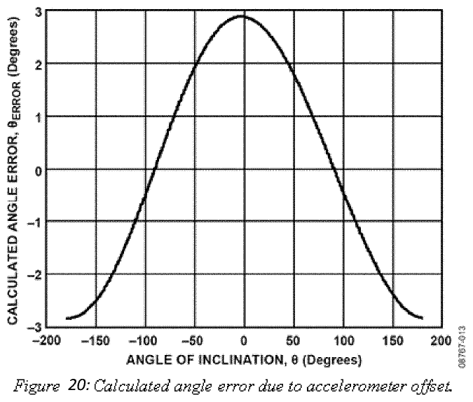

To demonstrate how large the error can be, imagine first a dual-axis solution with perfect sensitivity, but with a 50 mg offset on the x-axis. At 0° the x-axis reads 50 mg and the y-axis reads 1 g. The resulting calculated angle would be 2.9°, resulting in an error of 2.9°. At ±180° the x-axis would report 50 mg, whereas the y-axis would report −1 g. This would result in a calculated angle and error of −2.9°. The error between the calculated angle and the actual angle is shown in FIG. 20 for this example. The error due to an offset may not only be large compared to the desired accuracy of the system, but it can vary, thus making it difficult to simply calibrate out an error angle. This becomes more complicated when an offset for multiple axes is included.

Effects of Sensitivity Mismatch Error

The main error component due to accelerometer sensitivity in a dual-axis inclination sensing application is when a difference in sensitivity exists between the axis of interest (as opposed to a single-axis solution, where any deviation between the actual sensitivity and the expected sensitivity results in an error). Because the ratio of the x- and y-axes is used, most of the error is cancelled if the sensitivities are the same.

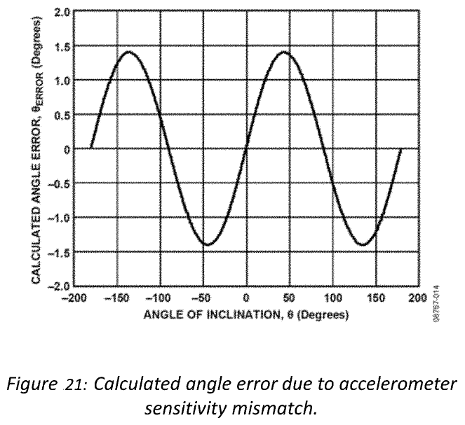

As an example of the effect of accelerometer sensitivity mismatch, assume that a dual-axis solution is used with perfect offset trim, perfect sensitivity on the y-axis, and +5 percent sensitivity on the x-axis. This means that in a 1 g field, the y-axis reports 1 g, whereas the x-axis reports 1.05 g. FIG. 21 shows the error in the calculated angle due to this sensitivity mismatch. Similar to offset error, the error due to accelerometer sensitivity mismatch varies over the entire range of rotation, making it difficult to compensate for the error after calculation of the inclination angle. Skewing the mismatch further by varying the y-axis sensitivity results in even greater error.

Basic Calibration Techniques

When the errors due to offset and sensitivity mismatch are combined, the error can become quite large and well beyond the acceptable limits in an inclination sensing application. To reduce this error, the offset and sensitivity should be calibrated and the calibrated output acceleration used to calculate the angle of inclination. When including the effects of offset and sensitivity, the accelerometer output is as follows:

A_{OUT[g]}=A_{OFF}+(Gain×A_{ACTUAL}) (14)

where:

A_{OFF} is the offset error, in g.

Gain is the gain of the accelerometer, ideally a value of 1.

A_{ACTUAL} is the real acceleration acting on the accelerometer and the desired value, in g.

A simple calibration method is to assume that the gain is 1 and to measure the offset. This calibration then limits the accuracy of the system to the uncalibrated error in sensitivity. The simple calibration method can be done by placing the axis of interest into a 0 g field and measuring the output, which would be equal to the offset. That value should then be subtracted from the output of the accelerometer before processing the signal. This is often referred to as no-turn or single-point calibration, because the typical orientation of a device puts the x- and y-axes in the 0 g field. If a three-axis device is used, at least one turn or second point should be included for the z-axis.

A more accurate calibration method is to use two points per axis of interest (up to six points for a three-axis design). When an axis is placed into a +1 g and −1 g field, the measured outputs are as follows:

A_{+1g}[g]=A_{OFF}+(1g×Gain) (15)

A_{-1g}[g]=A_{OFF}−(1g×Gain) (16)

where the offset, A_{OFF}, is in g.

These two points can be used to determine the offset and gain as follows:

$$\begin{matrix}{A}_{OFF} [ g ] = 0.5 \times ( {A}_{+ 1 g} + {A}_{- 1 g} ) & ( 17 ) \\ Gain = 0.5 \times ( \frac{{A}_{+ 1 g} - {A}_{- 1 g}}{1 g} ) & ( 18 )\end{matrix}$$

where the offset, A_{OFF}, is in g.

This type of calibration also helps to minimize cross-axis sensitivity effects as the orthogonal axes are in a 0 g field when making the measurements for the axis of interest. These values would be used by first subtracting the offset from the accelerometer measurement and then dividing the result by the gain.

$${A}_{ACTUAL} [ g ] = \frac{{A}_{OUT} - {A}_{OFF}}{Gain} \qquad ( 19 )$$

where A_{OUT} and A_{OFF} are in g.

The calculations of A_{OFF} and gain in Equation 15 through Equation 19 assume that the acceleration values, A_{+1g} and A_{-1g} are in g. If acceleration in mg is used, the calculation of A_{OFF} in Equation 17 remains unchanged, but the calculation of gain in Equation 18 should be divided by 1,000 to account for the change in units.

The foregoing methods, used to convert from accelerometer data to joint angle data may be augmented by methods described in the following reference, incorporated herein by reference: Contents Tilt Sensing Using a Three-Axis Accelerometer by Mark Pedley, Freescale Semiconductor Document Number: AN3461 Application Note Rev. 6, March 2013 © 2007-2009, 2012-2013 Freescale Semiconductor, Inc.

### FURTHER EMBODIMENTS

This acceleration value, coupled with a constant gravity vector, can be used to convert the acceleration in a particular x, y, or z-direction to a specific angle (position).

In the normal human gait, the knee and ankle joints move in the z-direction (x is laterally, and y is longitudinal). Thus, the acceleration in the z-direction is obtained from the accelerometer (the x and y acceleration values are consequently disregarded.) As previously explained, the acceleration in the z-direction is converted to an accurate angular representation of the position of the knee or ankle joint in any stage of the gait. This means that two accelerometers, one placed on the shin and one placed on the foot, can measure the position of the knee and ankle joint. Two accelerometers on the healthy leg, one directly below the knee joint (shin) and one directly on the dorsal side of the foot measure the angular position of the knee and ankle joint respectively. These angles are sent wirelessly to a microcontroller. The microcontroller is programmed with previously measured gait analysis data for the entire gait cycle (normal, human, standardized right/left knee/ankle joint data recorded from a human gait analysis book.) The microcontroller pairs the angular data received from the accelerometer and the preprogrammed data. The pairing enables the synergy between the healthy leg and the prosthesis. These normal angle positions of where the knee and ankle joints should be are sent to two servo motors on the prosthetic, one controlling the knee joint and one controlling the ankle joint, which will move the prosthetic to the correct knee and ankle angular positions relative to the healthy leg and the human gait.

What is claimed is:

1. A prosthesis system comprising:
a knee sleeve comprising at least one sensor and configured to be worn on a healthy leg;
an ankle sleeve comprising at least one sensor and configured to be worn on a healthy ankle;
a prosthesis comprising a knee joint assembly and an ankle joint assembly;
a microcontroller;
at least one servomotor coupled to the microcontroller and to the prosthesis; and
communications circuitry for communicating data from sensors to the microcontroller;
wherein the microcontroller is configured to control motion of the prosthesis responsive to the data from the sensors.
